(19) United States
(12) Patent Application Publication
(10) Pub. No.: US 2015/0079004 A1
(43) Pub. Date: Mar. 19, 2015

(54) METHODS FOR INCREASING SWALLOWING EFFICACY

(71) Applicant: NESTEC S.A., Vevey (CH)

(72) Inventors: Michael Jedwab, Lausanne (CH); Adam Burbidge, Arzier (CH); Kala Marie Kaspar, Lausanne (CH); Julie Ann Swanson, Minneapolis, MN (US)

(73) Assignee: NESTEC S.A., Vevey (CH)

(21) Appl. No.: 14/397,240

(22) PCT Filed: Apr. 19, 2013
(86) PCT No.: PCT/EP13/58193

§ 371 Date: Oct. 27, 2014

(30) Foreign Application Priority Data
Apr. 27, 2012 (US) 61639584

(51) Int. Cl. A61K 31/723; A23L 2/52; A23L 1/054

(52) U.S. Cl. CPC A61K 31/723; A23L 1/0541; A23L 2/52; A23V 2002/00
USPC 424/9.6; 514/54; 426/2

(57) ABSTRACT

In a general embodiment, a method for increasing efficacy of a swallow response is provided. The method includes providing a thickened composition having a xanthan gum thickening component, and orally administering the composition to an individual having, or at risk of having, a swallowing impairment. In an embodiment, the efficacy of the swallow response is increased by reducing an amount of pharyngeal residue in the individual's piriform sinuses, valleculae, and/or pharyngeal wall during or after the individual swallows the composition.

### BACKGROUND

The present disclosure is directed to methods for increasing swallowing efficacy. More specifically, the present disclosure is directed to methods for increasing the efficacy of a swallow response by reducing the presence of pharyngeal residue in patients having, or at risk for having, a swallowing impairment.

Dysphagia is the medical term for the symptom of difficulty in swallowing and refers to any deglutition (swallowing) disorder, which may include, for example, abnormalities within the oral, pharyngeal and esophageal phases of swallowing. Many complications can occur as a result of swallowing dysfunctions including, for example, pneumonia, dehydration, malnutrition, airway obstruction, dysfunctional immune response, etc. Dysphagia affects individuals of all ages, but it tends to be both more serious and more prevalent in older individuals.

Many of the serious complications from dysphagia result from food or drink entering the trachea (windpipe) and lungs, also known as aspiration. Aspiration can occur during the act of swallowing, or may occur after swallowing (postdeglutitive aspiration). Postdeglutitive aspiration generally occurs as a result of pharyngeal residue that is leftover in the pharynx after swallowing.

Generally, swallowing is divided into phases, such as the oral phase and the pharyngeal phase. The goal of dysphagia treatment is to accomplish both safe and efficacious swallowing. Safe swallowing is determined with reference to the amount of aspiration or penetration (past the vocal cords) of food or drink during the oral or pharyngeal phase. During the oral phase, efficacious swallowing can be determined, for example, with reference to the amount of impaired lip closure, piecemeal deglutition (more than one swallow needed per bolus), and oral residue. During the pharyngeal phase, efficacious swallowing can be determined, for example, with reference to the amount of pharyngeal residue.

Thus, it would be desirable to have a method to increase the efficacy of swallowing by reducing the amount of pharyngeal residue.

### SUMMARY

In a general embodiment, a method for increasing efficacy of a swallow response is provided. The method includes providing a thickened composition comprising a xanthan gum thickening component, and orally administering the composition to an individual having or at risk of having a swallowing impairment.

In an embodiment, the efficacy of the swallow response is increased by reducing an amount of pharyngeal residue in the individual's piriform sinuses, valleculae, and/or pharyngeal wall during or after the individual swallows the composition. The pharyngeal residue may be measured with a video fluoroscope during or after the individual swallows the composition.

In an embodiment, the method includes administering a second composition to the individual wherein the second composition includes a starch-based thickening agent, and comparing the amount of pharyngeal residue in the individual's piriform sinuses, valleculae, and/or pharyngeal wall during or after the individual swallows the thickened composition to the amount of pharyngeal residue in the individual's piriform sinuses, valleculae, and/or pharyngeal wall during or after the individual swallows the second composition.

In an embodiment, the composition is administered to the individual in an edible carrier. The edible carrier may be a liquid carrier selected from the group consisting of water, milk, orange juice, coffee, tea, soda, or combinations thereof

In an embodiment, the composition includes the xanthan gum thickening component in an amount ranging from about 1.0 g to about 7.0 g per 100 mL of liquid carrier.

In an embodiment, the composition is administered to the individual in a bolus amount ranging from about 5 mL to about 20 mL.

In another embodiment, a method for reducing an amount of pharyngeal residue in an individual having, or at risk of having, a swallowing impairment is provided. The method includes orally administering to the individual a thickened composition including a xanthan gum thickening component in an amount ranging from about 1.0 g to about 7.0 g per 100 mL of liquid carrier.

In yet another embodiment, a method for treating an individual having, or at risk of having, a swallowing impairment is provided. The method includes orally administering to an individual a thickened composition including a xanthan gum thickening component in an amount ranging from about 1.0 g to about 7.0 g per 100 mL of liquid carrier, wherein the oral administration increases an efficacy of the individual's swallow response.

An advantage of the present disclosure is to provide methods for treating patients having, or at risk of having, a swallowing impairment.

Another advantage of the present disclosure is to provide methods for improving the swallowing efficacy of patients having, or at risk of having, a swallowing impairment.

Yet another advantage of the present disclosure is to provide methods for promoting safe and efficacious swallowing of food boluses.

Still another advantage of the present disclosure is to provide methods for increasing swallowing efficacy by reducing the presence of pharyngeal residue during and/or after swallowing.

Additional features and advantages are described herein, and will be apparent from the following Detailed Description and the figures.

### BRIEF DESCRIPTION OF THE FIGURES

FIG. 1 shows experimental results conducted using a composition including a starch-based thickening component in accordance with Example 1.

FIG. 2 shows experimental results in accordance with Example 1, conducted using a composition including a xanthan gum thickening component in accordance with the present disclosure.

### DETAILED DESCRIPTION

The present disclosure relates to increasing the efficacy of the swallow response for patients suffering from, or at risk for, swallowing impairments including, for example, dysphagia. The present disclosure also relates to methods for reducing an amount of pharyngeal residue in an individual's piriform sinuses, valleculae, and/or pharyngeal wall during or after swallowing.

As used herein, “efficacy” and “efficiency” are used interchangeably and are intended to have the same meaning.

As used herein, “impaired swallowing efficacy” or “impaired efficacy of a swallow response” means the presence of an impaired labial seal, oral residue, or piecewise deglutition during the oral phase, or the presence of pharyngeal residue during the pharyngeal phase.

As used herein, “impaired swallowing safety” or “impaired safety of a swallow response” means the presence of aspiration or penetration during deglutition.

As used herein, “pharyngeal residue” refers to a residual material that exceeds a normal, thin mucosal coating in the valleculae, piriform sinuses, and/or pharyngeal wall after swallowing.

As used herein, “swallowing efficacy” or “efficacy of a swallow response” means the lack of presence of an impaired labial seal, oral residue, or piecewise deglutition during the oral phase, or the lack of presence of pharyngeal residue during the pharyngeal phase.

As used herein, “swallowing safety” or “safety of a swallow response” means the lack of presence of aspiration or penetration during deglutition.

The normal swallowing of a human (or mammal) involves three distinct phases which are interdependent and well coordinated: (i) the oral, (ii) the pharyngeal, and (iii) the esophageal phases. In the oral phase, which is under voluntary control, food that has been chewed and mixed with saliva is formed into a bolus for delivery by voluntary tongue movements to the back of the mouth, into the pharynx. The pharyngeal phase is involuntary and is triggered by a food/liquid bolus passing through the faucial pillars into the pharynx. Contraction of the three constrictors of the pharynx propels the bolus towards the upper esophageal sphincter. Simultaneously, the soft palate closes the nasopharynx. The larynx moves upwards to prevent food or liquid passing into the airway, which is aided by the backward tilt of the epiglottis and closure of the vocal folds. The esophageal phase is also involuntary and starts with the relaxation of the upper esophageal sphincter followed by peristalsis, which pushes the bolus down to the stomach.

Dysphagia refers to the symptom or diagnosis of difficulty in swallowing. Esophageal dysphagia affects a large number of individuals of all ages, but is generally treatable with medications and is considered a less serious form of dysphagia. Oral pharyngeal dysphagia, on the other hand, is a very serious condition and is generally not treatable with medication. Oral pharyngeal dysphagia also affects individuals of all ages, but is more prevalent in older individuals.

Oral pharyngeal dysphagia is often a consequence of an acute event, such as a stroke, brain injury, or surgery for oral or throat cancer. In addition, radiotherapy and chemotherapy may weaken the muscles and degrade the nerves associated with the physiology and nervous innervation of the swallow reflex. It is also common for individuals with progressive neuromuscular diseases, such as Parkinson's Disease, to experience increasing difficulty in swallowing initiation. Representative causes of oropharyngeal dysphagia include those associated neurological, infectious illnesses, metabolic illnesses, myopathic illnesses, iatrogenic illnesses, and structural illnesses.

Severity of dysphagia may vary from: (i) minimal (perceived) difficulty in safely swallowing foods and liquids, (ii) an inability to swallow without significant risk for aspiration or choking, and (iii) a complete inability to swallow. Commonly, the inability to properly swallow foods and liquids may be due to food boluses being broken up into smaller fragments, which may enter the airway (e.g., aspiration) or leave unwanted residues in the oropharyngeal and/or esophageal tract during the swallowing process. If enough material enters the lungs, it is possible that the patient may drown on the food/liquid that has built up in the lungs. Even small volumes of aspirated food may lead to bronchopneumonia infection, and chronic aspiration may lead to bronchiectasis and may cause some cases of asthma.

“Silent aspiration,” a common condition among elderly, refers to the aspiration in the lack of a cough reflex. This occurs particularly when the dysphagic patient does not have an efficacious swallow, and there is significant buildup of pharyngeal residues. People may compensate for less-severe swallowing impairments by self-limiting their diet. The aging process itself, coupled with chronic diseases such as hypertension or osteoarthritis, predisposes the elderly to dysphagia that may go undiagnosed and untreated until a clinical complication such as pneumonia, dehydration, malnutrition (and related complications) occurs.

The economic costs of dysphagia are associated with hospitalization, re-hospitalization, infections, rehabilitation, loss of work time, clinic visits, use of pharmaceuticals, labor, care taker time, childcare costs, quality of life, increased need for skilled care. Dysphagia and aspiration impact quality of life, morbidity and mortality. Twelve-month mortality is high among individuals in institutional care who have dysphagia and aspiration.

Pneumonia is a common clinical consequence of dysphagia. The condition often requires acute hospitalization and emergency room visits. Similar to pneumonia, dehydration is a life-threatening clinical complication of dysphagia. Dehydration is a common co-morbidity among hospitalized individuals with neurodegenerative diseases such as Alzheimer's disease, Parkinson's disease, and multiple sclerosis (thus, likely to have a swallowing impairment).

Malnutrition and related complications (e.g., urinary tract infections, pressure ulcers, increased severity of dysphagia, dehydration, functional decline and related consequences can arise when swallowing impairment leads to fear of choking on food and liquids, slowed rate of consumption, and self-limited food choices. If uncorrected, inadequate nutritional intake exacerbates dysphagia as the muscles that help facilitate normal swallow weaken as physiological reserves are depleted. In addition, malnutrition has serious implications for patient recovery.

Malnourished patients have longer length of hospital stay, are more likely to be re-hospitalized, and have higher costs for hospital care. Furthermore, malnutrition leads to unintentional loss of weight and predominant loss of muscle and strength, ultimately impairing mobility and the ability to care for oneself. With the loss of functionality, caregiver burden becomes generally more severe, necessitating informal caregivers, then formal caregivers, and then institutionalization.

Among persons with neurodegenerative conditions (e.g., Alzheimer's disease), unintentional weight loss (a marker of malnutrition) precedes cognitive decline. In addition, physical activity can help stabilize cognitive health. Thus, it is important to ensure nutritional adequacy among persons with neurodegenerative conditions to help them have the strength and endurance to participate in regular therapeutic exercise and guard against unintentional weight loss, muscle wasting, loss of physical and cognitive functionality, frailty, dementia, and progressive increase in caregiver burden.

Each of pneumonia, dehydration, and malnutrition are avoidable clinical complications of dysphagia.

Considering the prevalence of dysphagia, possible complications related thereto, and the costs associated with same, it would be beneficial to provide methods for promoting more efficacious swallowing of boluses in patients suffering from such swallowing disorders. Such methods would improve the lives of a large and growing number of persons with swallowing impairments. Specific interventions (e.g., to promote oral health, help restore normal swallow, or reinforce a swallow-safe bolus) can enable persons to eat orally (vs. being tube fed and/or requiring PEG placement) and experience the psycho-social aspects of food associated with general well being while guarding against the potentially negative consequences that result from lack of adequate swallowing ability. Improvements in the intake of nutrition by dysphagic patients may also enable such patients to swallow a wider variety of food and beverage products safely and comfortably, which may lead to an overall healthier condition of the patient and prevent further health-related decline.

Severe oral pharyngeal dysphagia may require nutrition to be supplied by tube feeding. Mild to moderate oral pharyngeal dysphagia may require the texture of foods to be modified in order to minimize the likelihood of choking or aspiration. This may include the thickening of liquids and/or pureeing of solid foods, both of which have been shown to be the most effective means of preventing choking and aspiration during the eating process.

Improving an individual's ability and efficiency to swallow improves the individual's safety through reduced risk of pulmonary aspiration. An efficient swallow may permit greater independence from feeding assistance and/or reduced length of time spent in feeding-assistance during meal consumption. All of these previously described factors are aimed at improving an individual's quality of life.

A known treatment to improve the swallowing safety for beverages and liquid foods is to increase the viscosity of the food/beverage by adding starch thickeners. Such thickening is thought to improve bolus control and timing of swallowing. It is, however, often disliked by patients because of the extra swallowing effort, and is also known to leave residues at high levels of viscosity.

For solid foods, pureed diets are often prescribed when problems with mastication and swallowing of solid pieces occur in patients. However, these pureed diets may lack the natural cohesiveness that saliva provides to “real” food boluses. Thus, thickening of such purees may be desired, while at the same time, the residue deposits are disfavored.

Applicants have surprisingly found that the administration of a thickened composition including a xanthan gum thickening component increases the efficacy of a swallow response by decreasing the presence of pharyngeal residue while at least maintaining swallowing safety. As discussed above, the reduction of pharyngeal residue assists in reducing the possibility of harmful conditions such as postdiglutitive aspiration and/or silent aspiration.

In a general embodiment, the present disclosure provides a method for increasing efficacy of a swallow response. The method includes providing a thickened composition comprising a xanthan gum thickening component, and orally administering the composition to an individual having or at risk of having a swallowing impairment.

In the context of this disclosure, xanthan gum is food grade and can be commercially obtained from numerous suppliers. Xanthan gum is a high molecular weight, long chain polysaccharide composed of the sugars glucose, mannose, and glucuronic acid. The backbone is similar to cellulose, with added side chains of trisaccharides.

In an embodiment, the compositions contain xanthan gum in an amount ranging from about 0.5 g to about 8 g, about 1 g to about 7 g, about 2 g to about 6 g, or about 3 g to about 4 g, per every 100 mL of a liquid carrier (e.g., water). In an embodiment, the compositions contain xanthan gum in an amount ranging from about 1.2 g to about 6 g.

The compositions may be administered in a bolus amount ranging from of about 3 mL to about 30 mL, about 4 mL to about 25 mL, about 6 mL to about 15 mL, or about 7 mL to about 10 mL. In an embodiment, the composition is administered in a bolus amount ranging from about 5 mL to about 20 mL.

By administering the xanthan gum-containing composition, the efficacy of a swallow response in patients having or at risk of a swallowing response may be improved, while at least maintaining the swallowing safety. This may enable such patients to swallow a wider variety of food and beverage products safely and comfortably. Such advantages may be achieved by reducing the presence of pharyngeal residue, which enables the patient to consume a variety of products without the uncomfortable throat coating and fear of later aspiration.

In an embodiment, the xanthan gum-containing composition is administered to the individual in an edible carrier. The edible carrier may be any suitable food, solid carrier, liquid carrier, or combinations thereof In an embodiment, the edible carrier may be a liquid carrier selected from the group consisting of water, milk, orange juice, coffee, tea, soda, or combinations thereof The skilled artisan will appreciate, however, that possible liquid carriers are not limited to the examples disclosed herein and may include any liquid carrier. In another embodiment, the edible carrier may be an oral nutritional supplement.

In an embodiment, the swallowing efficacy of the xanthan gum-containing composition is measured by reference to a composition containing a starch-based thickening component. In an embodiment, the swallowing efficacy of the xanthan gum-containing composition is measured by reference to any known thickened composition.

In an embodiment, the swallowing efficacy of the xanthan gum-containing composition is measured using a video fluoroscopy procedure. Video fluoroscopy is a lateral video x-ray procedure. Generally, the patient is asked to swallow compositions which include contrast media, such as barium sulfate, in a variety of different viscosities and swallowing positions. The results of a video fluoroscopy generally provide a set of prescribed swallowing positions and preferred viscosities deemed to encourage safe and efficacious swallowing for that specific patient.

By way of example and not limitation, the following example is illustrative of an embodiment of the present disclosure.

### EXAMPLE

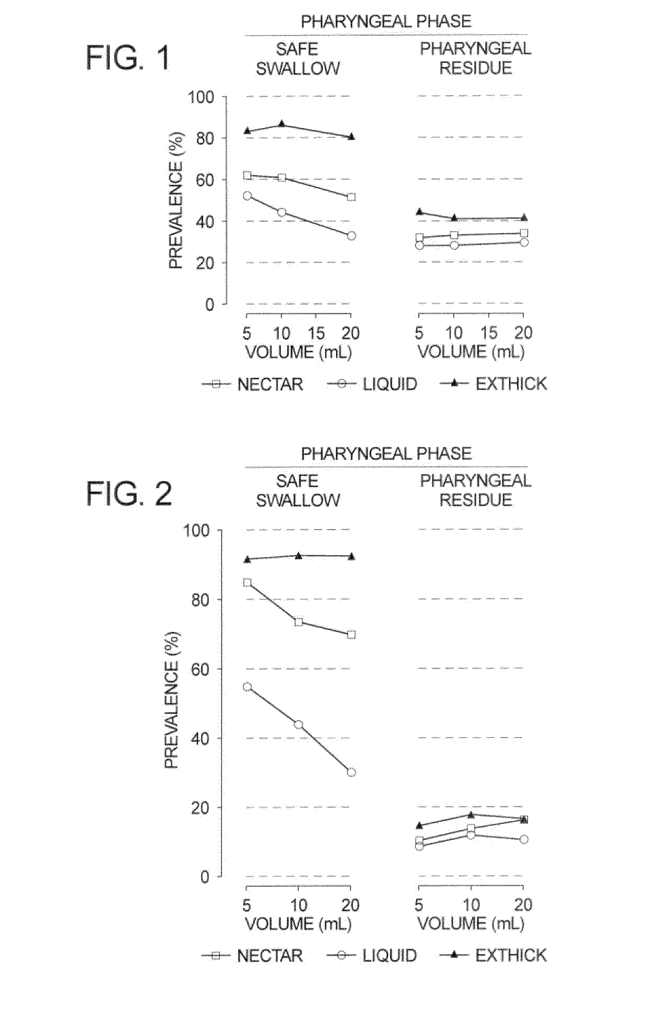

Applicants previously performed video fluoroscopy swallowing experiments using a composition containing a starch-based thickening component. Specifically, bolus volumes of 5, 10 and 20 mL were administered to patients in viscosities ranging from nectar (about 295.02 mPa·s at about 25° C.), liquid (about 21.61 mPa·s at about 25° C.), and pudding (3682.21 mPa·s at about 25° C.). The results of these experiments are shown in FIG. 1. It can be seen by reference to FIG. 1 that approximately 40% of the tested patients experienced pharyngeal residue at all viscosity levels. More specifically, the percentages of patients that experienced pharyngeal residue ranges from about 30% (for liquid viscosity boluses) to about 46% (for pudding viscosity boluses).

Applicants have also performed substantially similar video fluoroscopy swallowing experiments using a composition containing a xanthan gum thickening component in accordance with the present disclosure. The results of these experiments are shown at FIG. 2. It can be seen by reference to FIG. 2 that Applicants have surprisingly found that less than approximately 18% of patients experienced pharyngeal residue at all viscosity levels when the xanthan gum-containing composition is administered.

Specifically, for bolus volumes of 5 mL, 10 mL and 20 mL at liquid viscosity, approximately 7%, 10% and 9% of patients experienced pharyngeal residue. For bolus volumes of 5 mL, 10 mL and 20 mL at nectar viscosity, approximately 9%, 12% and 15% of patients experienced pharyngeal residue. For bolus volumes of 5 mL, 10 mL and 20 mL at extra thick viscosity, approximately 13%, 17% and 15% of patients experienced pharyngeal residue.

The less than 18% of patients experiencing pharyngeal residue with the xanthan gum-containing composition is in contrast to the approximately 40% of patients experiencing pharyngeal residue with the compositions containing a starch-based thickening component. Thus, Applicants have surprisingly found that the administration of a xanthan gum-containing composition improves swallowing efficacy by reducing the presence of pharyngeal residue.

Methods and procedures for the experiments using the compositions containing the starch-based thickening component may be found at Clavé, P, et al., “Accuracy of the volume-viscosity swallow test for clinical screening of oropharyngeal dysphagia and aspiration,” Clinical Nutrition (2008), doi:10.1016/j.clnu.2008.06.011. The methods and procedures for the experiments using the compositions containing the xanthan gum thickening component were similar or the same in all material aspects.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

1. A method for increasing efficacy of a swallow response comprising:
providing a thickened composition comprising a xanthan gum thickening component; and
orally administering the composition to an individual having, or at risk of having, a swallowing impairment.

2. The method of claim 1, wherein increasing the efficacy of the swallow response comprises reducing an amount of pharyngeal residue in the individual's piriform sinuses, valleculae, and/or pharyngeal wall during or after the individual swallows the composition.

3. The method of claim 1 comprising measuring with a video fluoroscope an amount of pharyngeal residue in the individual's piriform sinuses, valleculae, and/or pharyngeal wall during or after the individual swallows the composition.

4. The method of claim 3 comprising administering a second composition to the individual wherein the second composition comprises a starch-based thickening component, and comparing an amount of pharyngeal residue in the individual's piriform sinuses, valleculae, and/or pharyngeal wall during or after the individual swallows the xanthan gum thickening composition to an amount of pharyngeal residue in the individual's piriform sinuses, valleculae, and/or pharyngeal wall during or after the individual swallows the second composition.

5. The method of claim 1, wherein the composition is administered to the individual in an edible carrier.

6. The method of claim 5, wherein the edible carrier is a liquid carrier selected from the group consisting of water, milk, orange juice, coffee, tea, soda, and combinations thereof.

7. The method of claim 1, wherein the composition comprises the xanthan gum thickening component in an amount ranging from about 1.0 g to about 7.0 g per 100 mL liquid carrier.

8. The method of claim 7, wherein the composition is administered to the individual in a bolus amount ranging from about 5 mL to about 20 mL.

9. A method for reducing an amount of pharyngeal residue in an individual having, or at risk of having, a swallowing impairment, the method comprising:
orally administering to the individual a thickened composition comprising a xanthan gum thickening component in an amount ranging from about 1.0 g to about 7.0 g per 100 mL liquid carrier.

10. The method of claim 9, wherein the pharyngeal residue is located in the individual's piriform sinuses, valleculae, and/or pharyngeal wall during or after the individual swallows the composition.

11. The method of claim 9, wherein the composition is administered to the individual in an edible carrier.

12. The method of claim 11, wherein the edible carrier is a liquid carrier selected from the group consisting of water, milk, orange juice, coffee, tea, soda, and combinations thereof.

13. The method of claim 10 further comprising measuring the amount of pharyngeal residue with a video fluoroscope.

14. The method of claim 10, wherein the composition is administered to the individual in a bolus amount ranging from about 5 mL to about 20 mL.

15. A method for treating an individual having, or at risk of having, a swallowing impairment, the method comprising:
orally administering to the individual a thickened composition comprising a xanthan gum thickening component in an amount ranging from about 1.0 g to about 7.0 g per 100 mL liquid carrier, wherein the oral administration increases an efficacy of the individual's swallow response.

16. The method of claim 15, wherein increasing the efficacy of the swallow response comprises reducing an amount of pharyngeal residue in the individual's piriform sinuses, valleculae, and/or pharyngeal wall during or after the individual swallows the composition.

17. The method of claim 16 comprising measuring the amount of pharyngeal residue with a video fluoroscope.

18. The method of claim 15, wherein the composition is administered to the individual in an edible carrier.

19. The method of claim 18, wherein the edible carrier is a liquid carrier selected from the group consisting of water, milk, orange juice, coffee, tea, soda, and combinations thereof.

20. The method of claim 15, wherein the composition is administered to the individual in a bolus amount ranging from about 5 mL to about 20 mL.
